(19) United States
(12) Patent Application Publication
(10) Pub. No.: US 2018/0042814 A1
(43) Pub. Date: Feb. 15, 2018

(54) BIDIRECTIONAL DIAGNOSIS AND THERAPY DEVICE USED IN COMBINATION WITH SMART EQUIPMENT

(71) Applicant: INNO HEALTH TECHNOLOGY CO., LTD., Taichung (TW)

(72) Inventor: Chih-Cheng Chiang, Taichung (TW)

(21) Appl. No.: 15/236,998

(22) Filed: Aug. 15, 2016

(51) Int. Cl. A61H 39/00; A61B 5/00

(52) U.S. Cl. CPC A61H 39/002; A61B 5/4854; A61B 5/4836; A61B 5/6898; A61H 2201/5023; A61H 2201/5015; A61H 2201/5012; A61H 2201/1223

(57) ABSTRACT

The present invention is a smart equipment with bidirectional diagnosis and therapy device, comprising a power supply unit, an input unit, a microprocessor, a digital signal converter, a signal transmission unit, a high-voltage diagnosis and therapy unit and a smart equipment. Generally, the power supply unit is used for providing required power, while the digital signal converter is used for connecting the signal transmission unit and the smart equipment, such that a diagnosis and therapy signal sent back from the high-voltage diagnosis and therapy unit is acquired by the smart equipment. The smart equipment is loaded with application software for processing the diagnosis and therapy signal correspondingly, and then enable the user to manipulate the high-voltage diagnosis and therapy unit for diagnosis and therapy via the input unit according to suggestion from application software. Thus, correct diagnosis and therapy is allowed for the user to achieve the best effect.

### FIELD OF THE INVENTION

The present invention is related to an electronic medical appliance, particularly to an electronic medical appliance outputting high-voltage electromagnetism for stimulating acupoints on two hands and any other acupoints.

### BACKGROUND OF THE INVENTION

Referring to FIGS. 1 and 2, there are shown schematic diagrams of acupoints on a hand and feet corresponding to viscera and organs of human body, in which reflex zones of acupoints 1 refer to acupuncture points, such as acupoint for sciatic nerve 2, acupoint for cold 3 and etc., for parts of human body. In accordance with traditional Chinese medicine, stimulating acupuncture points for parts of human body by acupuncture is capable of treating diseases correspondingly, which is widely used and accepted by the public. Acupuncture, however, is an invasive therapy, and should not be performed without medical license. Moreover, the needles used for acupuncture are generally reused by multiple users. Although these needles are disinfected, infection may be caused if disinfection is not completely or contamination occurs during storage, and then dread is resulted. Moreover, fainting during acupuncture may occur due to repulsion of acupuncture, because of physiological uniqueness.

Referring to Taiwan patent publication no. M507277, therefore, there is disclosed a device controlling function of electronic acupuncture by smartphone, comprising a smartphone, an electronic acupuncture appliance and an electronic acupuncture-controlling means, in which the electronic acupuncture-controlling means is used for controlling the operation of the electronic acupuncture appliance, so as to perform acupuncture on acupuncture points for parts of human body. Moreover, an operation and display interface of the electronic acupuncture-controlling means is integrally displayed on the touch screen of the smartphone to show the locations of acupuncture points of human body and textual guidance of acupuncture on the touch screen of the smartphone, such that manipulating acupuncture by a user via the smartphone is allowed.

The device as described above is a portable electronic acupuncture appliance, which produces the effect of electrotherapy of stimulating various acupuncture points of human body by a voltage generated from a current. The conventional electronic acupuncture appliance, however, is used for therapy to be better late than never when pathological changes in the organs of human body have been found, but not for diagnosis and therapy for self-health care. Moreover, although the location diagram of acupuncture points of human body and the textual guidance of acupuncture are provided for reference, the problem of insufficient or excessive duration of acupuncture often occurs because it is generally incapable of determining the time duration, during which acupuncture should be performed, accurately for a non-professional user.

### SUMMARY OF THE INVENTION

Therefore, it is the main object of the present invention to provide a bidirectional diagnosis and therapy device used in combination with smart equipment, in which the smart equipment is loaded with application software, such that bidirectional diagnosis and therapy in accordance with suggestion and guidance of application software is allowed for a user to enhance the effect of acupuncture effectively.

The present invention is a bidirectional diagnosis and therapy device used in combination with smart equipment, comprising a power supply unit, an input unit, a microprocessor, a digital signal converter, a signal transmission unit, a high-voltage diagnosis and therapy unit and a smart equipment. In this case, the power supply unit is used for converting an external power supply into a required voltage to be supplied for each unit as required power. The input unit is provided for inputting operation commands. The digital signal converter is then used for converting a diagnosis and therapy signal of the high-voltage diagnosis and therapy unit into a digital signal, while the signal transmission unit is provided for transmitting the diagnosis and therapy signal.

Moreover, the high-voltage diagnosis and therapy unit is provided with an electronic oscillation generator, two output terminals and an electrode set. An input voltage to the electronic oscillation generator is used for generating an oscillating current outputted at the two output terminals. The electrode set is provided with two output points connected to the two output terminals, respectively. The microprocessor is used for connecting to the power supply unit, the input unit and the high-voltage diagnosis and therapy unit, as well as allowed for controlling the operation in accordance with the operation commands inputted from the input unit.

Furthermore, the smart equipment is connected to the signal transmission unit wirelessly, and loaded with application software processing the diagnosis and therapy signal correspondingly as well as being updated online. After the diagnosis and therapy signal is acquired, application software is then allowed for providing an usage suggestion with respect to the input unit in accordance with the diagnosis and therapy signal. The high-voltage diagnosis and therapy unit is manipulated for diagnosis and therapy by the user via the input unit in accordance with the usage suggestion.

Thereby, there are merits, in comparison with the prior art, of the present invention in that the user is allowed to perform diagnosis and therapy correctly via the usage suggestion, and application software may be updated online for providing the usage suggestion in response to the latest medical knowledge at any time, such that diagnosis and therapy in the latest and the most correct manner is allowed for the user to obtain the best diagnostic and therapeutic effect.

### BRIEF DESCRIPTION OF THE DRAWINGS

FIG. 1 is a schematic diagram of reflex zones of acupoints on the dorsum of the hand.

FIG. 2 is a schematic diagram of reflex zones of acupoints on the soles of the feet.

FIG. 3 is a functional block diagram of the present invention.

FIG. 4 is an external view of the structure of the present invention.

FIG. 5 is a functional block diagram of a high-voltage diagnosis and therapy unit of the present invention.

FIG. 6 is a circuit diagram of an electronic oscillation generator of the present invention.

FIG. 7 is a diagram showing an electrode set of another embodied mode of the present invention.

FIG. 8 is a diagram showing an electrode set of a further embodied mode of the present invention.

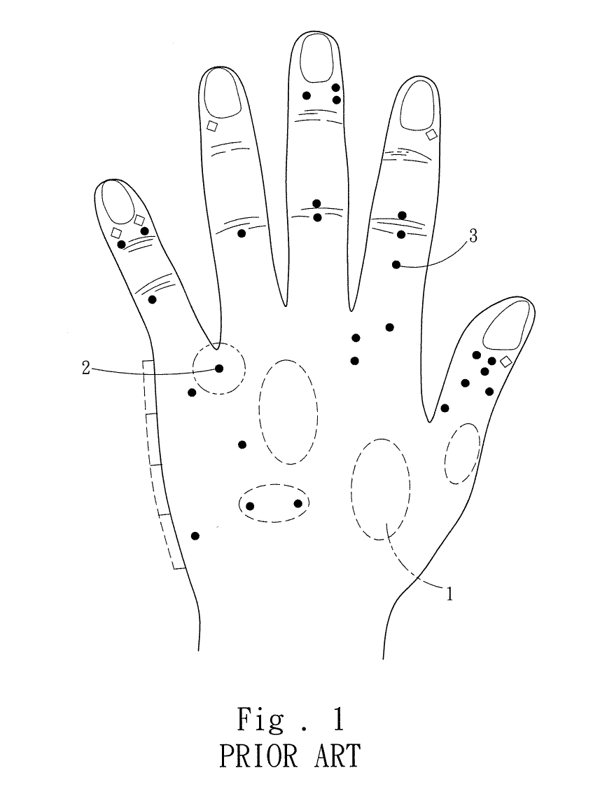

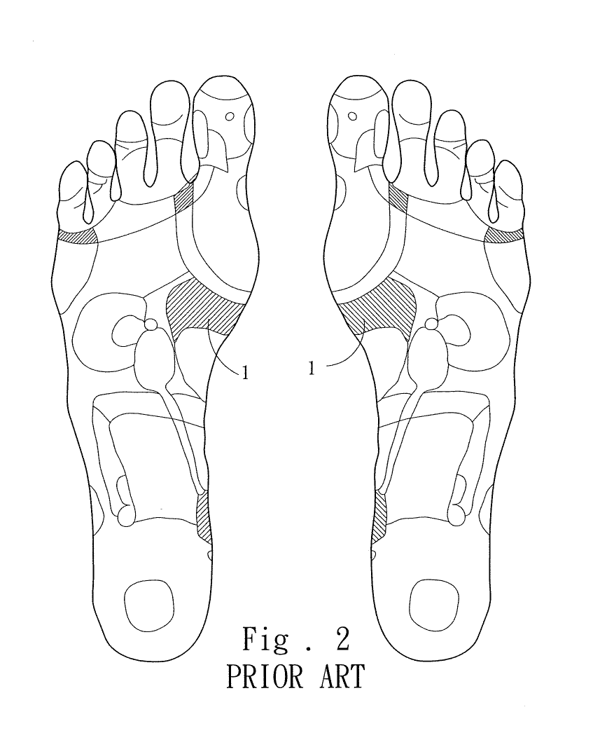

### DETAILED DESCRIPTION OF THE PREFERRED EMBODIMENTS

For better understanding and identifying the features, objects and effects of the present invention, one embodiment will be exemplified and explained in accompany with drawings as follows.

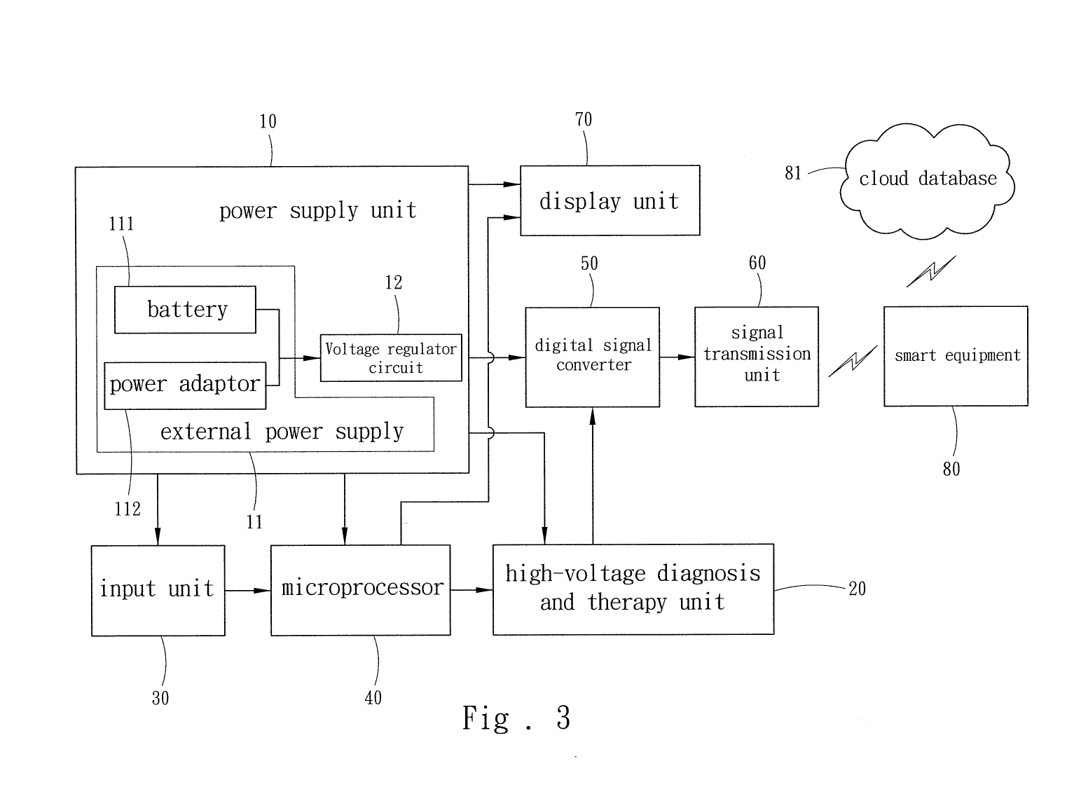

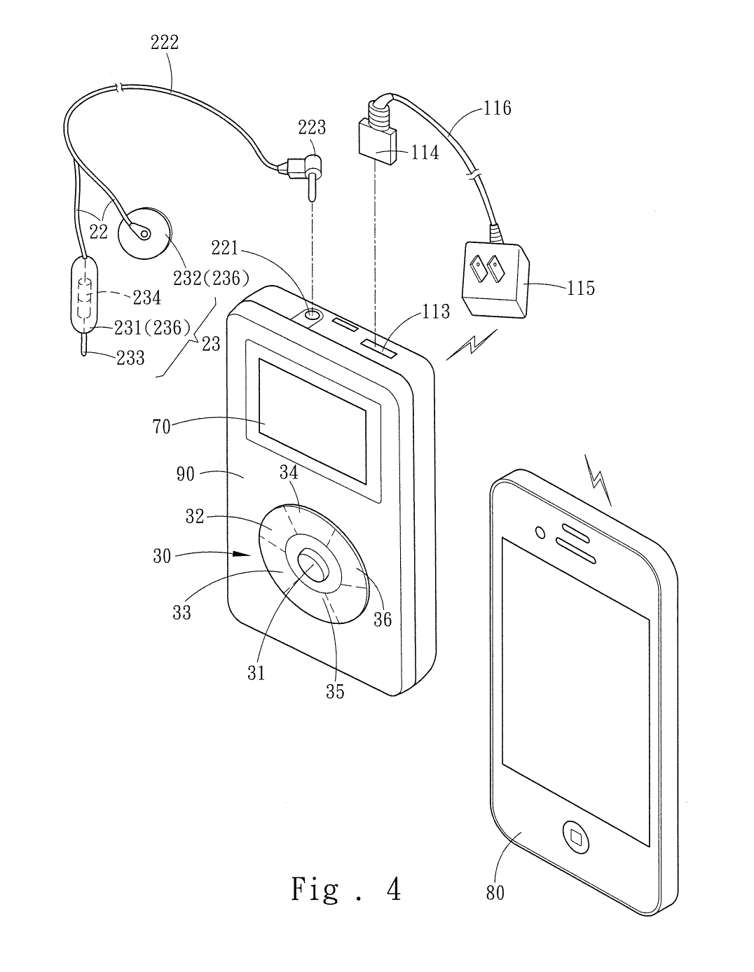

Referring to FIGS. 3 and 4, the present invention is a bidirectional diagnosis and therapy device used in combination with smart equipment, comprising a power supply unit 10, a high-voltage diagnosis and therapy unit 20, an input unit 30, a microprocessor 40, a digital signal converter 50, a signal transmission unit 60, a display unit 70 and a smart equipment 80. In this case, the power supply unit 10, the high-voltage diagnosis and therapy unit 20, the input unit 30, the microprocessor 40, the digital signal converter 50, the signal transmission unit 60 and the display unit 70 are installed in a housing 90 to constitute the bidirectional diagnosis and therapy device.

The power supply unit 10 is used for converting an external power supply 11 into a required voltage to be supplied for each unit as required power. The input unit 30 is provided for inputting operation commands. The digital signal converter 50 is then used for converting a diagnosis and therapy signal of the high-voltage diagnosis and therapy unit 20 into a digital signal, while the signal transmission unit 60 is provided for transmitting the diagnosis and therapy signal. Moreover, the display unit 70 is used for displaying related message to be looked up by a user.

The external power supply 11 may be either a battery 111 or a power adaptor 112. The battery 111 is portable, while the power adaptor 112 is capable of providing long-lasting power supply. The housing 90 is provided with a power jack 113 electrically connected to a power connector 114, such that power may be acquired from the mains socket (not shown in the figures) via a power cable 116 between the power connector 114 and a mains plug 115. Moreover, for ensuring safety of the circuit, the power supply unit 10 is further provided with a voltage regulator circuit 12 for providing a stable voltage supply.

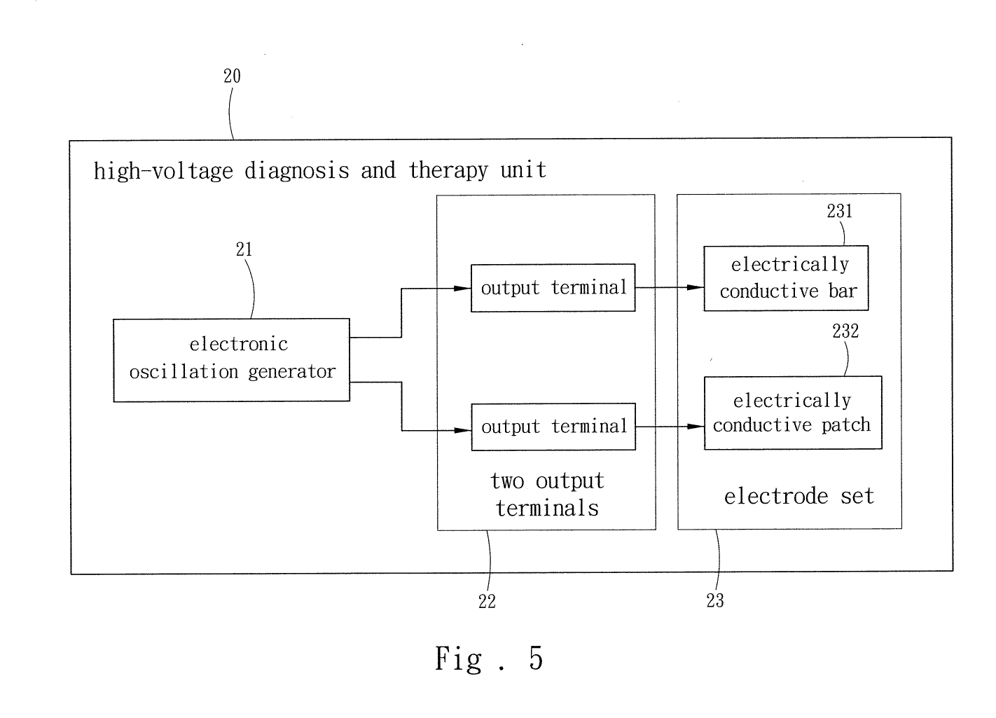

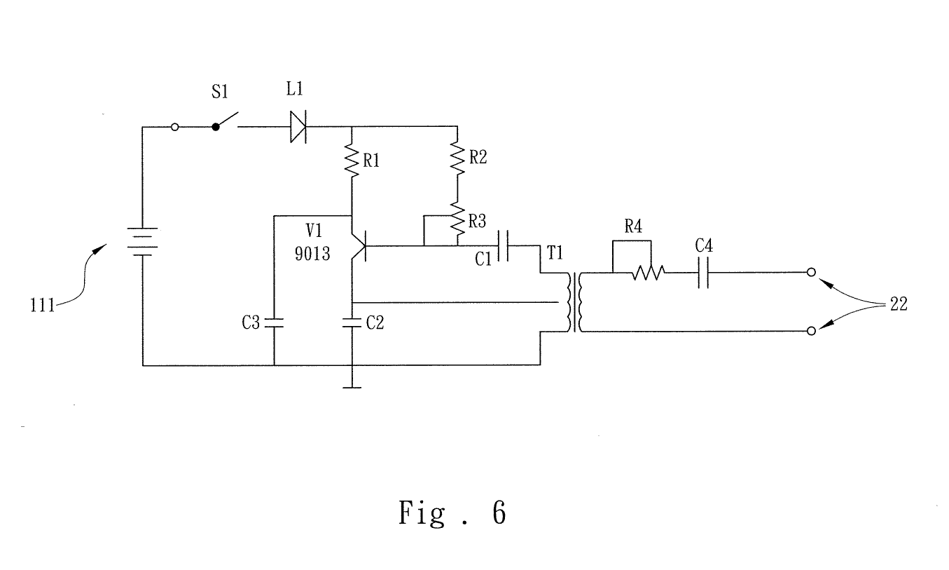

Referring to FIGS. 5 and 6 together, the high-voltage diagnosis and therapy unit 20 is provided with an electronic oscillation generator 21, two output terminals 22 and an electrode set 23. An input voltage to the electronic oscillation generator 21 is used for generating an oscillating current outputted at the two output terminals 22, in which the power source is generated by the battery 111 or the power source is generated by the power adaptor 112 converting alternating current (AC) into direct current (DC). Subsequently, a switch S1 for switching the power source and a light-emitting device L1 indicating whether internal operation is normal are used, followed by generating an oscillating current through a number of resistors R1, R2, variable resistors R3, R4, capacitors C1, C2, C3, C4, and a transistor V1 and transformer T1. Then, the oscillating current is outputted at the two output terminals 22 through the electronic oscillation generator 21.

The electrode set 23 is provided with two output points 236, which may be the combination of an electrically conductive bar 231 and an electrically conductive patch 232 as well as are connected to the two output terminals 22, respectively. The two output terminals 22 are connected to the electrically conductive bar 231 and the electrically conductive patch 232 via an output port 221 on the housing 90, as well as a connector 223 and a Y-shaped connecting wire 222. The leading end of the electrically conductive bar 231 is an electrode 233 made of conductive magnetic material. On the back of the electrode 233, there are provided with a number of magnets 234, by which the current outputted from the output terminal 22 is converted into magnetic wave and then directed to the electrode 233 to be outputted at the leading end of the electrically conductive bar 231.

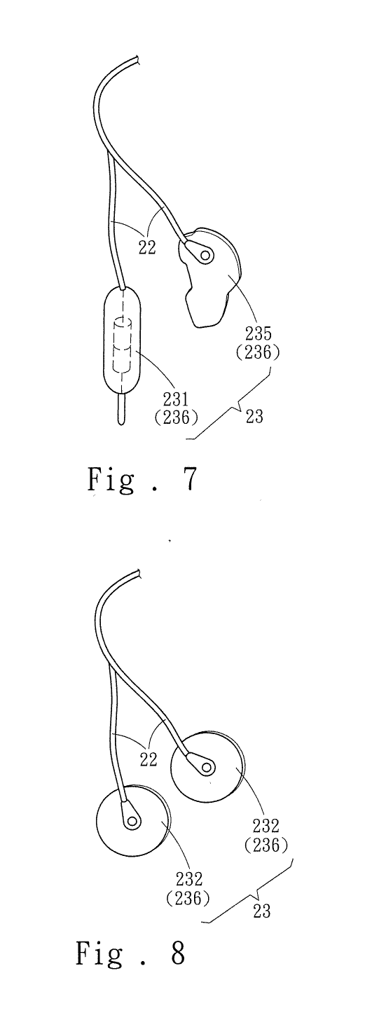

Furthermore, referring to FIGS. 7 and 8, the two output points 236 of the electrode set 23 may be also the combination of the electrically conductive bar 231 and an electrically conductive patch for pinna 235, similarly connected to the two output terminals 22, respectively. The electrically conductive patch for pinna 235 is worn on the ear of human body. The two output points 236 of the electrode set 23, otherwise, may be also the combination of at least two (preferably, 2 to 4) electrically conductive patches 232, which are similarly connected to the two output terminals 22, respectively. The electrically conductive patches 232 are provided for being adhered to human body.

In addition, the input unit 30 comprises a power switch 31, a positive button 32, a negative button 33 and a time-set button 34 for controlling the operation of the high-voltage diagnosis and therapy unit 20. Furthermore, the input unit 30 further comprises a diagnosis button 35 and a therapy button 36, which enable the high-voltage diagnosis and therapy unit 20 to provide outputs of specific strengths for diagnosis and therapy, respectively, switched by a user in the pressing manner. It is possible for the user to find out where therapy is required firstly in the diagnostic manner, and then the mode is switched for therapy in the therapeutic manner.

The microprocessor 40 is used for connecting to the power supply unit 10, the high-voltage diagnosis and therapy unit 20, the display unit 70 and the input unit 30. The microprocessor 40 is the core of the whole system. The microprocessor 40 is allowed for controlling the operation of various units in accordance with the operation commands inputted from the input unit 30, so as to meet individualized requirement of the user.

Furthermore, the smart equipment 80 is connected to the signal transmission unit 60 wirelessly. The smart equipment 80 may be any one of a smartphone, a tablet, etc. The signal transmission unit 60 may be a communication element using wireless technology, such as WIFI, Bluetooth, etc. Moreover, the smart equipment 80 is loaded with application software processing the diagnosis and therapy signal correspondingly as well as being updated online. After the diagnosis and therapy signal is acquired, application software is then allowed for providing an usage suggestion with respect to the input unit 30 in accordance with the diagnosis and therapy signal. The high-voltage diagnosis and therapy unit 20 is manipulated for diagnosis and therapy by the user via the input unit 30 in accordance with the usage suggestion. Application software of the present invention is installed in the smart equipment 80, and thus, may be updated to the latest version via the smart equipment 80 at any time. Therefore, the usage suggestion received by the user is the latest and the most correct one. Furthermore, in addition to finding out where therapy is required in the diagnostic manner in the present invention, the usage suggestion is further provided through application software-based processing in accordance with the past medical history and physical condition of the user. The best therapeutic effect may be achieved when the mode is switched to a therapeutic mode for therapy by the user, because the usage suggestion is made with reference to the diagnostic result as well as the past medical history and physical condition of the user.

Furthermore, several derivative applications are further provided when the present invention is combined with the smart equipment 80. For instance, the smart equipment 80 may be further connected to a cloud database 81, in which information of diagnosis and therapy may be stored. The information of diagnosis and therapy may be uploaded to the cloud database 81 by the smart equipment 80, and then monitored by specialized medical personnel. Once anxiety about deteriorating health is found, the user may be informed, at the earliest possible time, of further examination at medical institutions. Thus, health status of the user is monitored at any time.

As described above, the present invention enables the user to perform diagnosis and therapy correctly via the usage suggestion proposed by application software installed in a smart equipment. Therefore, application software may be updated online immediately, and then, allowed for providing the usage suggestion in response to the latest medical knowledge at any time. Thus, diagnosis and therapy in the latest and the most correct manner is allowed for the user to obtain the best diagnostic and therapeutic effect.

To sum up, the forgoing is only a preferred embodiment of the present invention, and not used for limiting the scope of the present invention. That is to say, various variations and modifications made in accordance with the patent claims should fall within the technical scope of the present invention.

What is claimed is:

1. A bidirectional diagnosis and therapy device used in combination with smart equipment, comprising:
a power supply unit, converting an external power supply into a required voltage;
a high-voltage diagnosis and therapy unit, provided with an electronic oscillation generator, two output terminals and an electrode set, an input voltage to said electronic oscillation generator being used for generating an oscillating current outputted at said two output terminals, said electrode set being provided with two output points, said two output points being connected to said two output terminals, respectively;
an input unit, provided for inputting operation commands;
a microprocessor, connected to said power supply unit, said input unit and said high-voltage diagnosis and therapy unit, as well as allowed for controlling operation in accordance with said operation commands inputted from said input unit;
a digital signal converter, converting a diagnosis and therapy signal of said high-voltage diagnosis and therapy unit into a digital signal;
a signal transmission unit, transmitting said diagnosis and therapy signal; and
a smart equipment, connected to said signal transmission unit wirelessly, and loaded with an application software processing said diagnosis and therapy signal correspondingly as well as being updated online, the application software being then allowed for providing an usage suggestion with respect to said input unit in accordance with said diagnosis and therapy signal after said diagnosis and therapy signal is acquired.

2. The bidirectional diagnosis and therapy device used in combination with smart equipment according to claim 1, wherein said external power supply is either a battery or a power adaptor.

3. The bidirectional diagnosis and therapy device used in combination with smart equipment according to claim 1, wherein said power supply unit is further provided with a voltage regulator circuit.

4. The bidirectional diagnosis and therapy device used in combination with smart equipment according to claim 1, wherein said input unit comprises a power switch, a positive button, a negative button and a time-set button.

5. The bidirectional diagnosis and therapy device used in combination with smart equipment according to claim 1, wherein said input unit comprises a diagnosis button and a therapy button, which enable said high-voltage diagnosis and therapy unit to provide outputs of specific strengths for diagnosis and therapy, respectively.

6. The bidirectional diagnosis and therapy device used in combination with smart equipment according to claim 1, wherein said two output points of said electrode set are presented as any one of combinations comprising an electrically conductive bar with an electrically conductive patch, said electrically conductive bar with an electrically conductive patch for pinna, and at least two electrically conductive patches, the leading end of said electrically conductive bar being an electrode made of conductive magnetic material, the back of said electrode being provided with a plurality of magnets, by which said current outputted from said output terminal is converted into magnetic wave and then directed to said electrode.

7. The bidirectional diagnosis and therapy device used in combination with smart equipment according to claim 1, wherein said smart equipment is further connected to a cloud database.

8. The bidirectional diagnosis and therapy device used in combination with smart equipment according to claim 1, further comprising a display unit connected to said microprocessor, said display unit displaying related message to be looked up by a user.

9. The bidirectional diagnosis and therapy device used in combination with smart equipment according to claim 8, wherein said power supply unit, said input unit, said microprocessor, said digital signal converter, said signal transmission unit, said high-voltage diagnosis and therapy unit and said display unit are installed in a housing.

10. The bidirectional diagnosis and therapy device used in combination with smart equipment according to claim 1, wherein said smart equipment is any one of a smartphone and a tablet.
